(19) United States
(12) Patent Application Publication
(10) Pub. No.: US 2021/0005285 A1
(43) Pub. Date: Jan. 7, 2021

(54) SYSTEM AND METHOD USING LOCAL UNIQUE FEATURES TO INTERPRET TRANSCRIPT EXPRESSION LEVELS FOR RNA SEQUENCING DATA

(71) Applicant: KONINKLIJKE PHILIPS N.V., EINDHOVEN (NL)

(72) Inventors: Jie Wu, Cambridge, MA (US); Yee Him Cheung, Boston, MA (US)

(21) Appl. No.: 16/979,444

(22) PCT Filed: Mar. 13, 2019
(86) PCT No.: PCT/EP2019/056354

§ 371 Date: Sep. 9, 2020

Related U.S. Application Data
(60) Provisional application No. 62/642,877, filed on Mar. 14, 2018.

(51) Int. Cl. G16B 30/10; G16B 50/30; G16B 50/10; G16B 40/00; G06F 16/907; G06F 16/903

(52) U.S. Cl. CPC G16B 30/10; G16B 50/30; G06F 16/90344; G16B 40/00; G06F 16/907; G16B 50/10

(57) ABSTRACT

A method (100) for characterizing gene transcript expression levels, comprising: (i) extracting (110) one or more unique features from each of a plurality of gene transcripts; (ii) storing (120) the extracted unique features in a unique feature database; (iii) receiving (130) a plurality of sequences sequenced from gene transcripts, wherein at least some of the sequences comprise one or more of the extracted unique features; (iv) comparing (140), by a processor, the plurality of sequences to the extracted unique features stored in the unique feature database; (v) identifying (150), based on a match between a sequence and an extracted unique feature, a gene transcript and/or gene from which the sequence was generated; and (vi) compiling (160) information about gene transcript expression levels based on said identified gene transcripts.

### FIELD OF THE DISCLOSURE

The present disclosure is directed generally to methods and systems for characterizing gene transcript expression levels using unique features in gene transcripts.

### BACKGROUND

RNA sequencing is an important tool for transcriptome study. This high-throughput technique offers several advantages compared to previous technologies, including the ability to detect novel and lowly expressed transcripts with broader dynamic ranges.

Protein diversity in eukaryotic organisms is largely increased by alternative splicing, which greatly increases transcriptome complexity. For example, it is estimated that more than 90% of multi-exon human genes experience alternative splicing, many of which are revealed by RNA sequencing data. The expression of these transcript variants are highly regulated and are differentially expressed across different tissues or developmental stages, and in tumors or diseases. As a result, estimating gene and transcript expressions from RNA sequencing data is a crucial element in basic and clinical bioinformatics research.

However, estimating gene and transcript expressions from RNA sequencing data is challenging. For example, since many genes express more than one transcript, allocating sequencing reads to the transcript from which they were derived is a major problem which any transcript expression estimation program must resolve. Other challenges include, for example, non-uniform distribution of the read coverage, among many others.

Current tools attempt to resolve the structures of the different expressed isoforms and estimate their expression levels based on RNA sequencing data. For example, some software can assemble RNA sequencing reads to a minimum number of transcripts in an attempt to identify all the fragments, and then utilizes a generative statistical model to estimate transcript abundances. Other analysis software maps the reads to the transcriptome directly instead of to the genome, and then uses a model to allocate reads to different isoforms.

However, these current tools do not solve all the challenges faced when analyzing RNA sequencing data. For example, tools typically examine entire RNA sequencing reads from the transcript start site to the transcript stop site, which is time consuming and computationally inefficient. Furthermore, as the complexity of resolving transcriptome structures increases, such as with small conditional RNA or low-quality RNA sequencing data, tools that rely on full RNA sequencing reads are less effective.

### SUMMARY OF THE DISCLOSURE

There is a continued need for tools that effectively and efficiently determine gene transcript expression levels from RNA sequencing data.

The present disclosure is directed to inventive methods and systems for characterizing gene transcript expression levels from RNA sequencing data. Various embodiments and implementations herein are directed to a system that extracts unique features from gene transcripts, including but not limited to unique exons, unique exon junctions, unique introns, unique start location, and/or unique stop locations, among others. The system receives or sequences gene transcripts and compares the sequences to the extracted unique features which are stored in a unique feature database. Based on matching between these sequences and extracted unique features, the system identifies the gene transcripts and compiles information about gene transcript expression levels.

Generally in one aspect, a method for characterizing gene transcript expression levels is provided. The method includes: (i) extracting one or more unique features from each of a plurality of gene transcripts; (ii) storing the extracted unique features in a unique feature database; (iii) receiving a plurality of sequences sequenced from gene transcripts, wherein at least some of the sequences comprise one or more of the extracted unique features; (iv) comparing, by a processor, the plurality of sequences to the extracted unique features stored in the unique feature database; (v) identifying, based on a match between a sequence and an extracted unique feature, a gene transcript from which the sequence was generated; and (vi) compiling information about transcript expression levels based on said identified gene transcripts.

According to an embodiment, the unique features comprise one or more of a unique exon, a unique exon junction, a unique intron, a unique start location, and/or a unique stop location.

According to an embodiment, comparing comprises aligning each of the plurality of sequences sequenced from gene transcripts with one or more unique features.

According to an embodiment, the method further includes the step of providing a sample for RNA sequencing.

According to an embodiment, the method further includes the step of sequencing gene transcripts from one or more cells to generate the plurality of sequences.

According to an embodiment, the method further includes the step of associating, in the unique feature database, at least some of the extracted unique features with annotation information.

According to an embodiment, the unique feature database comprises extracted unique features rather than full gene transcripts.

According to an embodiment, the identifying step comprises a probability that the identified gene transcript is the transcript from which the sequence was generated.

According to an embodiment, the sequence matches an extracted unique feature from two different genes, and the identifying step comprises identifying two or more gene transcripts from which the sequence was generated or might have been generated.

According to an aspect is a system for characterizing gene transcript expression levels. The system includes: a database of unique features extracted from each of a plurality of gene transcripts; a comparison module configured to: (i) compare a plurality of sequences sequenced from gene transcripts to the extracted unique features stored in the unique feature database; and (ii) identify, based on a match between a sequence and an extracted unique feature, a gene transcript from which the sequence was generated; and a compilation module configured to compile information about gene transcript expression levels based on said identified gene transcripts.

According to an embodiment, the system further includes a feature extraction module configured to extract the unique features from the plurality of gene transcripts. According to an embodiment, the feature extraction module is further configured to associate at least some of the extracted unique features with annotation information.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects of the various embodiments discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for characterizing gene expression levels, in accordance with an embodiment.

FIG. 2 is a schematic representation of transcript expression estimation using unique features of a gene transcript, in accordance with an embodiment.

FIG. 3 is a schematic representation of a system and method for gene or gene transcript expression level characterization, in accordance with an embodiment.

FIG. 4 is a schematic representation of a system for characterizing gene expression levels, in accordance with an embodiment.

### DETAILED DESCRIPTION OF EMBODIMENTS

The present disclosure describes various embodiments of a system and method for compiling information about gene transcript expression levels using unique features extracted from gene transcripts. More generally, Applicant has recognized and appreciated that it would be beneficial to provide a system that enables rapid and efficient characterization of gene transcript expression levels using RNA sequencing data. The system comprises a unique feature database which stores unique features extracted from gene transcripts, including but not limited to unique exons, unique exon junctions, unique introns, unique start location, and/or unique stop locations, among many other unique features. The system receives or sequences gene transcripts and compares the sequences to the extracted unique features in the unique feature database. If at least a portion of a sequence matches one or more extracted unique features, the gene transcript from which the sequence was generated is identified. In this way, the system can compile information about gene transcript expression levels from the source of the RNA sequencing data.

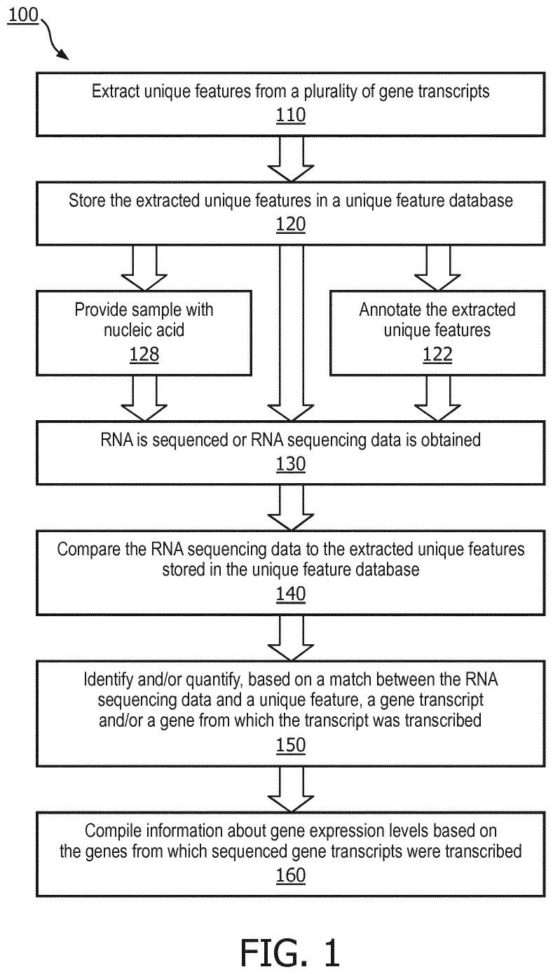

Referring to FIG. 1, in one embodiment, is a flowchart of a method 100 for characterizing gene transcript expression levels using RNA sequencing data. At step 110 of the method, unique features from gene transcripts are extracted. According to an embodiment, for most or all of the transcripts in a target or investigated transcriptome, the system can scan the transcripts obtained by sequencing and/or identified based on genetic analysis, and can compare these transcripts to identify unique features. The system may utilize only unique features that are found, based on this comparison, to result from transcription and/or alternative splicing from a single gene. Alternatively, the system may utilize unique features found to result from transcription and/or alternative splicing from two or more genes. There may be, for example, a threshold for determination of how many genes or alternative splices a feature may be found before and/or after which it will or will not be identified as a sufficiently unique feature for the methods described or otherwise envisioned herein.

A unique feature is a parameter of the RNA sequence that results from splicing of the gene from which the RNA is transcribed. In many cases, the parameter results from alternative splicing of the gene from which the RNA is transcribed. For example, a unique feature of a gene transcript may result from unique exons, which may be exons that are unique to a subset of transcripts from a gene. A unique feature of a gene transcript may result from unique exon junctions, which may be exon junctions that are unique to a subset of transcripts from one gene, such as from exon skipping among other processes. A unique feature of a gene transcript may result from unique intron retention events, which may result from one or more introns being retained in a transcript. A unique feature of a gene transcript may result from unique transcription start and/or stop sites, since different transcripts from a gene may begin and/or end at different locations along the gene.

As described herein, quantifying these unique identifiers can effectively resolve the deconvolution problems that typically result from RNA sequencing data. For example, even if degraded RNAs are sequenced, the expression of transcripts can still be evaluated accordingly as long as unique features are still covered by enough reads. Furthermore, the extracted unique features may comprise only a subset of the total information found within the entire transcriptome of the organism from which the RNA sequencing data is obtained. This further resolves many of the issues faced by existing systems and reduces the computing time significantly. It also enables rapid screening of a large volume of RNA sequencing data in a short period of time.

At step 120 of the method, the extracted unique features are stored in a unique feature database. The unique feature database may be part of the system, or may be located remote from the system. For example, the unique feature database may be a database or memory associated with a processor or other component of the system. Alternatively, the unique feature database may be a database or memory which is kept remotely from the system using the unique features to characterize RNA sequencing data. For example, the generated unique feature database may be utilized by one or more systems, some or all of which may be decentralized relative to the database or memory, to perform the analysis described or otherwise envisioned herein. Accordingly, the system may comprise or otherwise be in communication with a wired and/or wireless communications system facilitating communication between the system and the remote database or memory. The extracted unique features may be stored in the unique feature database for retrieval and downstream use, or may be stored in the unique feature database in a format enabling rapid search of and/or comparison or alignment of RNA sequencing data to the extracted unique features. According to an embodiment, the unique feature database comprises extracted unique features rather than full gene transcripts, which facilitates rapid identification of genes and/or gene transcripts.

At step 122 of the method, one or more of the unique features in the unique feature database are associated with annotation information. For example, a unique feature may be labeled, tagged, marked, or otherwise associated in memory with information about the gene from which it was extracted, and/or from the transcript from which it was extracted. The annotation information may comprise information about the location of the unique feature or associated transcript in the genome, information about the organism from which the unique feature was extracted, information about alternative splicing of the gene from which the unique feature was extracted, and/or any other information about the source of the unique feature, the location of the unique feature, and more.

At step 130 of the method, RNA is sequenced or RNA sequencing data is obtained. For example, RNA may be sequenced from a sample comprising or potentially comprising ribonucleic acid. According to an embodiment, therefore, at step 128 of the method a sample is provided for nucleic acid extraction and analysis. The sample may compose ribonucleic acid from one or more cells of one or more microorganisms such as bacteria, viruses, fungi, and/or from plants or animals, among many other sources. A sample may comprise ribonucleic acid molecules from one organism or from multiple organisms. Samples may be obtained in a clinical setting, from the environment, from indoor or outdoor surfaces, or from any other source. It is recognized that there is no limitation to the source of the sample, or the ribonucleic acid(s) in the sample. The sample and/or the ribonucleic acids therein may be prepared for sequencing using any method for preparation, which may be at least in part dependent upon the sequencing platform. According to an embodiment, the ribonucleic acids may be extracted, purified, and/or amplified, among many other preparations or treatments.

The system may comprise a sequencing platform configured to sequence at least a portion of a ribonucleic acid from a sample. Any method of and/or platform for sequencing ribonucleic acid may be utilized to obtain RNA sequencing data. Accordingly, the sequencing platform can be any sequencing platform, including but not limited to any systems described or otherwise envisioned herein. According to one embodiment the sequencing platform may comprise a controller or other analysis module for downstream analysis and characterization. According to another embodiment, the sequencing platform communicates the generated RNA sequencing data, in real-time or at certain time points, to a local or remote controller or other analysis module for downstream analysis and characterization.

Alternatively, the system may retrieve or otherwise receive RNA sequencing data from a remote sequencing platform, or from a database or memory comprising stored RNA sequencing data. For example, the system may be in communication with a local and/or remote database or memory comprising stored RNA sequencing data, or may receive an upload or other delivery of RNA sequencing data. Thus, the analysis described or otherwise envisioned herein may be performed as RNA sequencing data is obtained and/or may be obtained after RNA sequencing data is obtained.

At step 140 of the method, the system compares the sequenced or obtained sequences to the extracted unique features stored in the unique feature database. For example, the system may comprise a processor or other computing component configured or programmed to compare the sequenced or obtained sequences to the extracted unique features stored in the unique feature database. The comparison may be performed, for example, by aligning a sequenced or obtained sequence to one or more of the extracted unique features, either in the unique feature database or in a memory or processor.

According to an embodiment, the system may utilize an algorithm to compare sequenced or obtained sequences to extracted unique features. For example, splicing quantification algorithms such as SpliceTrap which quantifies exon inclusion levels using paired-end RNA sequencing data, or MISO (Mixture-of-Isoforms) which identifies differentially regulated isoforms or exons across samples, may optionally be modified for use. For example, splicing quantification algorithms can quantify known or novel alternative splicing events from RNA sequencing reads. These are applicable to quantifying the unique features, and can be used and/or modified to estimate the ratios and expressions of the unique features. Reads on exon junctions and distinctive regions can be important and the algorithms can be used to find the optimal solutions. According to an embodiment, a cassette exon may be alternatively skipped in certain transcripts, and its inclusion ratio and expression level can be investigated by examining the reads in middle exon(s) and/or in exon junctions.

At step 150 of the method, a gene transcript from which a sequence was generated is identified and/or quantified based on a match between a sequence and an extracted unique feature. According to an embodiment, there may be a threshold or probabilistic requirement for positive identification of a gene transcript, which may optionally be based on quality of unique feature(s) identified, quantity of unique features, and/or other parameters. According to an embodiment, the system quantifies the gene transcripts while identifying them, or in addition to identifying them. For example, the system counts, tracks, records, or otherwise quantifies identified gene transcripts, which facilitates information about gene transcript expression based on the relative expressions measured from the unique features. Splicing quantification algorithms, for example, may be used to quantify the gene transcripts.

According to an embodiment, a sequence matches one or more extracted unique feature from two or more different gene transcripts. For example, in some embodiments a short sequence may comprise a unique feature found in several different gene transcripts, but is missing additional sequence information that could differentiate between the full transcripts. Accordingly, identifying step 150 may comprise identifying two or more transcripts from which the sequence was generated or could have been generated. The system may be configured to only report transcript which can definitively defined, or can report sequences that potentially identify multiple transcripts.

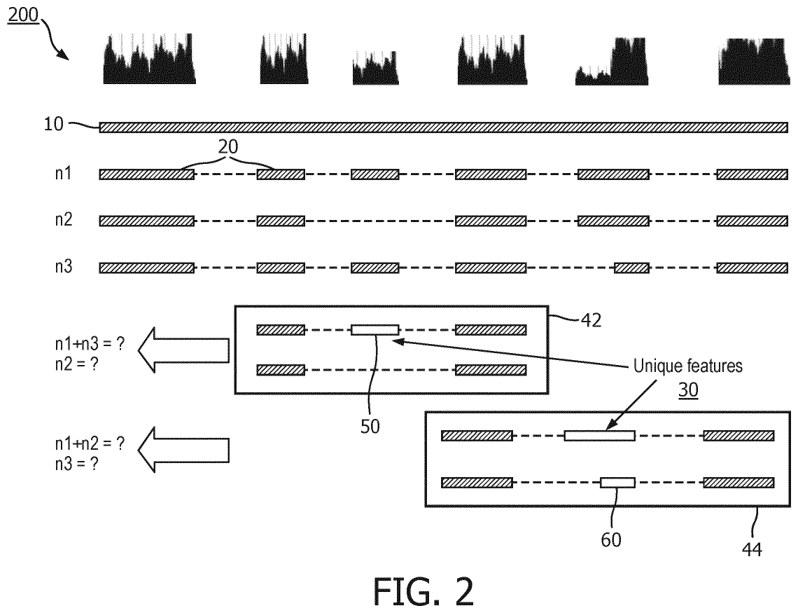

Referring to FIG. 2, in one embodiment, is a schematic representation 200 of transcript expression estimation using unique features of a gene transcript. The gene 10 includes at least three different transcripts (n1, n2, and n3), each of which includes a different set of exons 20. According to an embodiment, the three different transcripts of this gene can be discriminated by two unique features 30, one skipped exon 50 and one alternative splice site 60. For example, unique feature 50 is present in comparison 42, enabling identification of a read as being n2 versus n1 or n3. As another example, unique feature 60 is present in comparison 44, which enables identification of a read as being n3 versus n1 or n2. Expression of transcripts n1, n2, and n3 can be solved by looking at each feature separately and then combining the observations.

At step 160 of the method, the system compiles information about gene transcript and/or gene expression levels based on the identified gene transcripts and/or genes from the analyzed RNA sequences. According to an embodiment, the system may track, record, store, or otherwise count the specific gene transcript or gene as each sequence is identified in step 150 of the method. The transcript expression levels may be summarized in any format, including standard formats such as FPKM values among many other formats. Feature quantifications are collected and summarized to interpret transcript expression based on the relationships between the features and the transcripts. In complicated cases, a linear model can be used to solve the matrix. When there are conflicts between results summarized from different features due to un-even distribution of reads across the transcripts, certain representative values such as an average or maximum can be used. According to an embodiment, the compilation comprises annotation information from the unique feature database. According to an embodiment, the system may report transcript expression levels as or with probability information, including a probability that an identified transcript is the transcript from which the sequence was generated.

As described herein, the extracted unique features can be used as markers of certain gene transcript and/or gene expression profiles. Just one advantage of using the unique features is that they can combine views from both the gene level and the splicing level. Furthermore, quantifications of unique features from one gene can be used to model expression patterns of the transcripts from that gene. Indeed, this can be performed even without knowledge of the actual expression values of the transcripts.

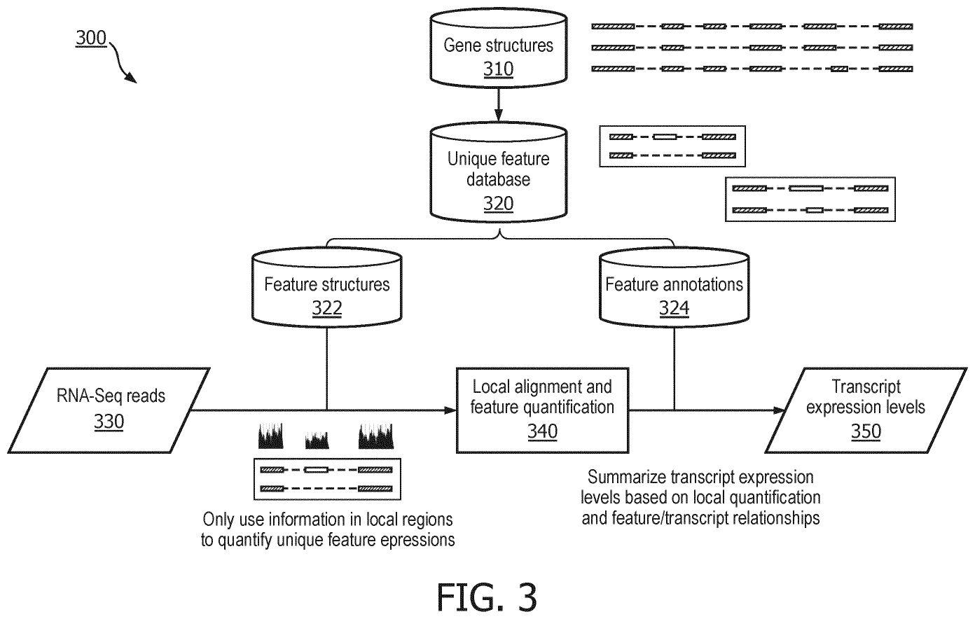

Referring to FIG. 3 is a schematic representation 300 of a system and method for gene transcript expression level characterization as described or otherwise envisioned herein. The system includes a unique feature database 320 comprising unique features 322 which are extracted from gene structures 310 as described or otherwise envisioned herein. The unique feature database 320 may also comprise one or features annotations 324 associated with the extracted unique features 322. A plurality of RNA sequencing reads 330 are obtained either by sequencing or by receiving sequencing data, and are compared at 340 to the extracted unique features 322 in the unique feature database 320. The transcript expression levels 350 are obtained by compiling, summarizing, or otherwise characterizing the genes and/or gene transcripts using the feature annotations in the unique feature database 320.

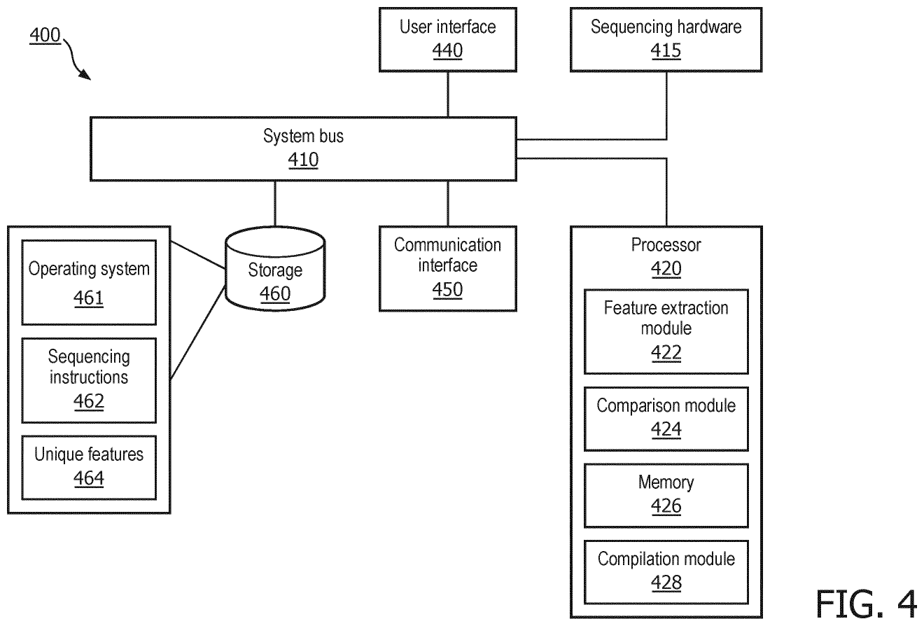

Referring to FIG. 4, in one embodiment, is a schematic representation of a system 400 for characterizing gene transcript expression levels. System 400 includes one or more of a processor 420, memory 426, user interface 440, communications interface 450, and storage 460, interconnected via one or more system buses 410. In some embodiments, such as those where the system comprises or implements a sequencer or sequencing platform, the hardware may include additional sequencing hardware 415, which may be any sequencer or sequencing platform. It will be understood that FIG. 4 constitutes, in some respects, an abstraction and that the actual organization of the components of the system 400 may be different and more complex than illustrated.

According to an embodiment, system 400 comprises a processor 420 capable of executing instructions stored in memory 426 or storage 460 or otherwise processing data. Processor 420 performs one or more steps of the method, and may comprise one or more of the modules described or otherwise envisioned herein. Processor 420 may be formed of one or multiple modules, and can comprise, for example, a memory 426. Processor 420 may take any suitable form, including but not limited to a microprocessor, microcontroller, multiple microcontrollers, circuitry, field programmable gate array (FPGA), application-specific integrated circuit (ASIC), a single processor, or plural processors.

Memory 426 can take any suitable form, including a non-volatile memory and/or RAM. The memory 426 may include various memories such as, for example a cache or system memory. As such, the memory 426 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 400. It will be apparent that, in embodiments where the processor implements one or more of the functions described herein in hardware, the software described as corresponding to such functionality in other embodiments may be omitted.

User interface 440 may include one or more devices for enabling communication with a user such as an administrator. The user interface can be any device or system that allows information to be conveyed and/or received, and may include a display, a mouse, and/or a keyboard for receiving user commands. In some embodiments, user interface 440 may include a command line interface or graphical user interface that may be presented to a remote terminal via communication interface 450. The user interface may be located with one or more other components of the system, or may located remote from the system and in communication via a wired and/or wireless communications network.

Communication interface 450 may include one or more devices for enabling communication with other hardware devices. For example, communication interface 450 may include a network interface card (NIC) configured to communicate according to the Ethernet protocol. Additionally, communication interface 450 may implement a TCP/IP stack for communication according to the TCP/IP protocols. Various alternative or additional hardware or configurations for communication interface 450 will be apparent.

Storage 460 may include one or more machine-readable storage media such as read-only memory (ROM), random-access memory (RAM), magnetic disk storage media, optical storage media, flash-memory devices, or similar storage media. In various embodiments, storage 460 may store instructions for execution by processor 420 or data upon which processor 420 may operate. For example, storage 460 may store an operating system 461 for controlling various operations of system 400. Where system 400 implements a sequencer and includes sequencing hardware 415, storage 460 may include sequencing instructions 462 for operating the sequencing hardware 415. According to an embodiment, storage 460 may include a unique feature database 464 which have been extracted pursuant to the methods described or otherwise envisioned herein.

It will be apparent that various information described as stored in storage 460 may be additionally or alternatively stored in memory 426. In this respect, memory 426 may also be considered to constitute a storage device and storage 460 may be considered a memory. Various other arrangements will be apparent. Further, memory 426 and storage 460 may both be considered to be non-transitory machine-readable media. As used herein, the term non-transitory will be understood to exclude transitory signals but to include all forms of storage, including both volatile and non-volatile memories.

While system 400 is shown as including one of each described component, the various components may be duplicated in various embodiments. For example, processor 420 may include multiple microprocessors that are configured to independently execute the methods described herein or are configured to perform steps or subroutines of the methods described herein such that the multiple processors cooperate to achieve the functionality described herein. Further, where system 400 is implemented in a cloud computing system, the various hardware components may belong to separate physical systems. For example, processor 420 may include a first processor in a first server and a second processor in a second server. Many other variations and configurations are possible.

According to an embodiment, processor 420 comprises one or more modules to carry out one or more functions or steps of the methods described or otherwise envisioned herein. For example, processor 420 may comprise a feature extraction module 422, comparison module 424, and/or compilation module 428. According to an embodiment, feature extraction module 422 analyzes genes and/or gene transcripts to identify one or more parameters of RNA sequences that result from splicing of the gene from which the RNA is transcribed, including but not limited to alternative splicing of the gene from which the RNA is transcribed. The unique features may be extracted using any process for feature identification from genes and/or gene transcripts. According to an embodiment, the system may utilize only unique features that are found to result from transcription and/or alternative splicing from a single gene. Alternatively, the system may utilize unique features found to result from transcription and/or alternative splicing from two or more genes. There may be, for example, a threshold for determination of how many genes or alternative splices a feature may be found before and/or after which it will or will not be identified as a sufficiently unique feature for the methods described or otherwise envisioned herein. Among many other features, the extracted unique feature may be result from unique exon junctions, unique intron retention events, unique transcription start and/or stop sites, and many others. Once extracted, the unique features may be stored in the unique feature database 464 or other memory. In some embodiments, the unique features are stored remotely from one or more other components of the system.

According to an embodiment, processor 420 comprises a comparison module 424. According to an embodiment, comparison module 424 compares the sequenced or obtained sequences to the extracted unique features stored in the unique feature database 464. The comparison may be performed, for example, by aligning an RNA sequence to one or more of the extracted unique features, either in the unique feature database or in a memory or processor. According to an embodiment, the system may utilize an algorithm to compare the sequences to extracted unique features. The comparison module 424 may identify a gene transcript from which the sequence was generated, and/or may identify a gene from which the gene transcript was transcribed, based on a match between a sequence and an extracted unique feature. According to an embodiment, there may be a threshold or probabilistic requirement for positive identification of a gene transcript and/or a gene, which may optionally be based on quality of unique feature(s) identified, quantity of unique features, and/or other parameters. The comparison module 424 may count, track, record, or otherwise quantify gene transcripts, which facilitates information about gene transcript expression based on the relative expressions measured from the unique features. The comparison module 424 may utilize splicing quantification algorithms to quantify the gene transcripts, among other methods.

According to an embodiment, processor 420 comprises a compilation module 428. According to an embodiment, compilation module 428 compiles or summarizes information about gene transcript and/or gene expression levels based on identified gene transcripts and/or identified genes from which the sequences were generated or transcribed. According to an embodiment, the system may track, record, store, or otherwise count the specific gene transcript or gene as each sequence is analyzed. The transcript expression levels may be summarized in any format, including standard formats such as FPKM values among many other formats. According to an embodiment, the compilation module 428 retrieves, compiles, and/or summarizes annotation information from the unique feature database associated with the identified gene transcripts and/or identified genes.

According to an embodiment, the system described or otherwise envisioned herein provides significant functional advantages over existing systems, in both efficiency and accuracy. For example, by improving the identification of gene transcripts, the system provides significant computational efficiency over existing systems. By using only information in small regions instead of all the reads from transcripts, gene expression estimation is simplified to quantifying local critical elements. This enables the system to perform improved high-throughput screening of RNA sequencing data.

According to another embodiment, the system described or otherwise envisioned herein improves existing systems by enabling determination of transcript expression levels from incomplete RNAs, which are common in low-quality RNA sequencing data and scRNA sequencing data. The approaches described herein avoid the bias that comes in from regions where transcription is very high or very low.

According to another embodiment, the system described or otherwise envisioned herein improves existing systems where unique features are correlated with phenotypes. Compared to gene expression, quantification of these features provides a higher resolution profile. It may be more robust too, as the unique features may be able to capture effects of unknown transcript variant since more detailed patterns can be revealed with these local measurements. Similarly, the unique features can be used as additional evidence to cluster RNA sequencing samples, such as for subpopulation inference of scRNA sequencing data among other processes.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for characterizing gene transcript expression levels, comprising:
extracting one or more unique features from each of a plurality of gene transcripts;
storing the extracted unique features in a unique feature database;
receiving the plurality of sequences generated from gene transcripts sequenced from one cell, wherein at least some of the sequences comprise one or more of the extracted unique features;
comparing, by a processor, the plurality of sequences to the extracted unique features stored in the unique feature database;
identifying, based on a match between a sequence and an extracted unique feature, a gene transcript from which the sequence was generated; and
compiling information about gene transcript expression levels based on said identified gene transcripts.

2. The method of claim 1, wherein the unique features comprise one or more of a unique exon, a unique exon junction, a unique intron, a unique transcription start location, and/or a unique transcription stop location.

3. The method of claim 1, wherein comparing comprises aligning each of the plurality of sequences with one or more unique features.

4. The method of claim 1, further comprising the step of quantifying the identified gene transcripts.

5. The method of claim 1, further comprising the step of sequencing gene transcripts from one or more cells to generate the plurality of sequences.

6. The method of claim 1, further comprising the step of associating, in the unique feature database, at least some of the extracted unique features with annotation information.

7. The method of claim 1, wherein the unique feature database comprises extracted unique features rather than full gene transcripts.

8. The method of claim 1, wherein said identifying step comprises a probability that the identified gene transcript is the transcript from which the sequences was generated.

9. The method of claim 1, wherein a sequence matches an extracted unique feature from two different gene transcripts, and said identifying step comprises identifying two or more gene transcripts from which the sequence was generated or might have been generated.

10. A system (400) for characterizing gene transcript expression levels, comprising:
A feature extraction module configured to extract unique features from each of a plurality of gene transcripts generated by sequencing gene transcripts from one cell;
a database of the unique features extracted from each of a plurality of gene transcripts;
a comparison module configured to: (i) compare a plurality of sequences sequenced from gene transcripts to the extracted unique features stored in the unique feature database; and (ii) identify, based on a match between a sequence and an extracted unique feature, a gene transcript and/or gene from which the sequence was generated; and
a compilation module configured to compile information about gene transcript expression levels based on said identified gene transcripts.

11. (canceled)

12. The system of claim 10, wherein the feature extraction module is further configured to associate at least some of the extracted unique features with annotation information.

13. The system of claim 10, wherein the unique features stored in the unique feature database comprise one or more of a unique exon, a unique exon junction, a unique intron, a unique start location, and/or a unique stop location.

14. The system of claim 10, wherein comparing comprises aligning each of the plurality of sequences with one or more unique features.

15. The system of claim 10, wherein a sequence matches an extracted unique feature from two different gene transcripts, and said identifying step comprises identifying two or more gene transcripts from which the sequence was generated or might have been generated.
